(19) United States
(12) Patent Application Publication
(10) Pub. No.: US 2007/0202503 A1
(43) Pub. Date: Aug. 30, 2007

(54) Pharmaceutical Composition Of (+)-Erythro-Mefloquine And Its Use

(76) Inventors: Helen Frances Baker, Essex (GB); Robin Mark Bannister, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/591,158

(22) PCT Filed: Mar. 17, 2005
(86) PCT No.: PCT/GB05/01014

§ 371 Date: Oct. 10, 2006

(30) Foreign Application Priority Data
Mar. 17, 2004 (GB) 0406014.1

(51) Int. Cl. C12Q 1/68

(52) U.S. Cl. 435/6

(57) ABSTRACT

A pharmaceutical composition is in the form of a unit dosage comprising 1 to 60 mg (+)-erythro-mefloquine. This is intended for daily dosing.

### FIELD OF THE INVENTION

This invention relates to a composition of (+)-erythro-mefloquine and to its use in the treatment of inflammatory disorders.

### BACKGROUND OF THE INVENTION

Mefloquine racemate (Lariam) is a known anti-malarial drug. It is typically formulated as a tablet comprising 250 mg of the active ingredient, to be taken weekly. Lariam has well known side-effects.

Bates et al, Int. Arch. Allergy Appl. Immunol. (1998) 86: 446-452, discloses that racemic mefloquine stimulates human neutrophil degranulation. Although the data show that mefloquine is pro-inflammatory, it is stated (without evidence) that mefloquine may have utility as an anti-inflammatory agent. Any such utility would be compromised, in chronic treatment, by the known adverse effects of Lariam, and especially in patients with cardiac disease.

WO02/19994 discloses for the first time that the single enantiomer (+)-erythro-mefloquine is useful in the treatment of chronic conditions, and in particular chronic inflammatory conditions such as osteoarthritis and rheumatoid arthritis. The publication reports that the given enantiomer has greatly reduced side-effects.

Inflammatory conditions have been treated with anti-TNF antibodies. It is known that several patients (as many as 40%) are refractory to this treatment.

### SUMMARY OF THE INVENTION

The present invention is based at least in part on the realisation that there is a therapeutic window that can be exploited in the treatment of, say, malaria and inflammatory conditions, using (+)-erythro-mefloquine. Accordingly, a novel pharmaceutical composition is in the form of a unit dosage comprising 1 to 60 mg (+)-erythro-mefloquine, substantially free of the opposite enantiomer. This dosage form is intended to be taken daily.

The use of (+)-erythro-mefloquine may be particularly valuable in combination with an anti-TNF antibody. Such anti-bodies complement the broad, moderate IL-I antagonist activity of (+)-erythro-mefloquine, and the combination can help overcome the problems associated with patients who do not respond to anti-TNF therapy (as described above). Accordingly, such combination therapy constitutes a further aspect of the present invention.

Another feature of using (+)-erythro-mefloquine is that the undesirable effect of an immunosuppressant such as methotrexate can be reduced whilst retaining efficacy. Combination or coadministration with such an agent is therefore a further aspect of the invention.

### DESCRIPTION OF PREFERRED EMBODIMENTS

Despite the fact that mefloquine is associated with a long half-life, the daily dosage proposed according to the invention reduces peaks and troughs in the concentration of the active material. Given this relatively uniform level of drug in the system of the patient being treated, the chances of successful therapy are increased.

The amount of the agent that should be administered can readily be determined by the skilled man, taking into account the usual factors such as the type of patient, the nature of the condition being treated, and the route of administration. The amount of enantiomer may be higher or the same as that for the racemate, or may be modified depending on the co-administration of other drugs.

The dosage of the active component can be lower than has been associated with the administration of Lariam. The daily dosage according to the invention may be at least 5 mg, and is often no more than 15, 20 or 40 mg. A relatively low dosage may be preferable for women.

For use in the invention, the active agent may be formulated, e.g. together with a carrier, excipient or diluent, and administered, by procedures that are known in the art, including those already proposed for the racemate. Suitable compositions will depend on the intended route of administration, which may be, for example, oral, topical, nasal, rectal, sublingual, buccal or transdermal. Sustained, delayed, timed or immediate release compositions may be used.

The formulation is preferably a unit dose, intended for daily administration. It may be, for example, a capsule, ampoule or, preferably, a tablet typically containing filler, compression aid, disintegrant, wetting agent and lubricant.

Conditions that may be treated include conditions involving cartilage destruction, inflammatory conditions and those mediated by IL-2 and IL-6, e.g. rheumatoid arthritis, asthma, psoriasis, psoriatic arthritis, Crohn's disease, irritable bowel syndrome and systemic lupus erythematosus. Other relevant conditions are ulcerative colitis, COPD and asthma. The patient may be disposed to CNS side-effects, and/or may be undergoing concomitant therapy with another drug, e.g. a TNF antibody or an immunosuppressant such as methotrexate.

The use of (+)-erythro-mefloquine can provide the desired therapeutic effect, without tissue destruction, and can be safely administered at a relatively high dosage. The desired enantiomer of mefloquine may be in at least 50%, 70%, 90%, 95% or 99% excess, with respect to any other. The active agent may be used in any active form, e.g. salt or non-salt.

The following studies provide evidence on which the present invention is based.

Combination

200 mg tablets of (+)-erythro-mefloquine were prepared, respectively containing (A) 4.5 mg, (B) 9 mg and (C) 18 mg of this agent (4.92 mg, 9.86 mg and 19.71 mg of the HCl salt). Each formulation additionally contained 76 mg microcrystalline cellulose, 7 mg povidone, 10 mg crospovidone, 2 mg sodium lauryl sulphate, 2 mg magnesium stearate and also lactose (98.07 mg, 93.14 mg and 87.29 mg, respectively, in A, B and C).

The formulations were used on a background of methotrexate therapy. Adverse events were observed with the following frequency:

| | |
|---|---|
| Placebo | 36.8% |
| A | 5.9% |
| B | 22.2% |
| C | 16.7% |

Thus a combination of (+)-erythro-mefloquine and methotrexate has lower adverse events than methotrexate alone.

Efficacy

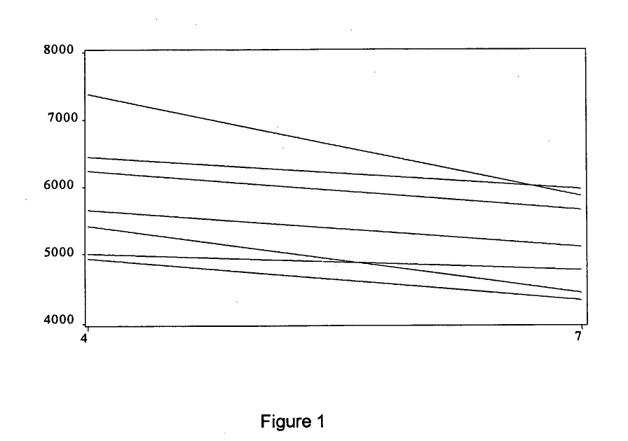

DAS28 scores (http://www.das-score.nl/www.das-score.nl/DAS CRP.html) for individual subjects were recorded for formulation B (9 mg (+)-erythro-mefloquine). The results are shown in FIG. 1, a plot of individual DAS score against Visit. A decrease in DAS score was observed for all patients; the average decrease was 0.71 units over the course of the study (1 month).

CNS Benefit

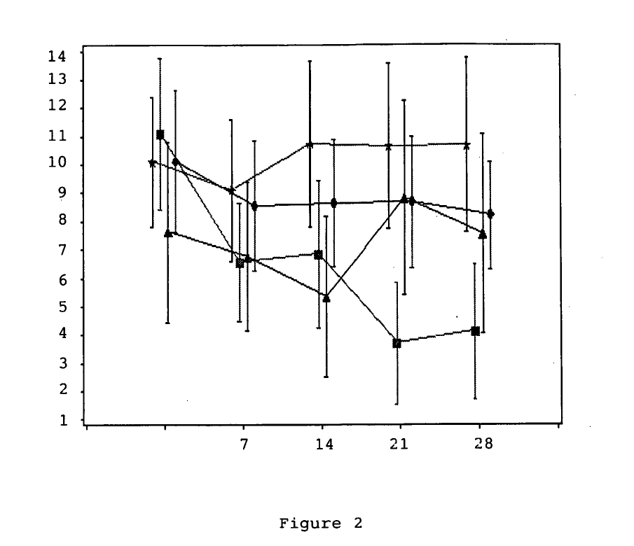

Racemic mefloquine shows a 7.5 unit increase in Total Mood Disturbance (TMD) on the Profile of Mood States Questionnaire when tested in a traveller study (van Riemskijk et al, Clin, Pharmacol. Ther. 2002: 72 294-301). In a clinical study, where patients who were taking a background therapy of methotrexate received either placebo or formulation A, B or C daily for 1 month, the latter decreased the TMD score (i.e. improved the mood of the patients). This is shown in FIG. 2, a plot of TMD (mean score) against time (days); ♦ represents placebo, ▴ represents A, ▪ represents B and ★ represents C.

PK Profile

Daily dosing with formulation C gave a minimum plasma concentration of 203 ng/ml and a maximum plasma concentration of 263 ng/ml, a difference of 60 ng/ml. A dose of 36 mg daily would equate to the usual racemic mefloquine dose. This could be expected to have a difference between minimum and maximum plasma concentration of about 120 ng/ml which is significantly different to the variation in plasma concentration seen with weekly dosing of racemic mefloquine, which is about 500 ng/ml.

1. A pharmaceutical composition in the form of a unit dosage comprising 1 to 60 mg (+)-erythro-mefloquine, substantially free of the opposite enantiomer.

2. The composition according to claim 1, wherein the unit dosage is a tablet comprising a carrier and/or excipient.

3. The composition according to claim 1, wherein the unit dosage comprises up to 40 mg (+)-erythro-mefloquine.

4. The composition according to claim 3, wherein the unit dosage comprises up to 20 mg (+)-erythro-mefloquine.

5. The composition according to claim 3, wherein the unit dosage comprises up to 15 mg (+)-erythro-mefloquine.

6. The composition according to claim 1, wherein the unit dosage comprises at least 5 mg (+)-erythro-mefloquine.

7. A method for the treatment of an inflammatory condition wherein said method comprises administering, to a subject in need of such treatment, a pharmaceutical composition in the form of a unit dosage comprising 1 to 60 mg (+)-erythro-mefloquine, substantially free of the opposite enantiomer.

8. The method according to claim 7, wherein the condition is osteoarthritis.

9. The method according to claim 7, wherein the condition is rheumatoid arthritis.

10. The method according to claim 7, wherein the condition is also treated with an anti-TNF antibody.

11. The method according to claim 7, wherein the subject of treatment is also receiving an immunosuppressant.

12. The method according to claim 11, wherein the immunosuppressant is methotrexate.

13. A product comprising (+)-erythro-mefloquine and an anti-TNF antibody, as a combined preparation for simultaneous, separate or sequential use in the treatment of an inflammatory condition.

14. A product comprising (+)-erythro-mefloquine and an immunosuppressant as a combined preparation for simultaneous, separate or sequential use in the treatment of an inflammatory condition and where immunosuppression is also required.

15. The product according to claim 14, wherein the immunosuppressant is methotrexate.
